(19) United States
(12) Patent Application Publication
(10) Pub. No.: US 2003/0008313 A1
(43) Pub. Date: Jan. 9, 2003

(54) Process for enhanced molecular target detection using layered rolling circle amplification

(76) Inventor: Richard S. Wiltshire, Southington, CT (US)

Correspondence Address: CARELLA, BYRNE, BAIN, GILFILLAN,, CECCHI, STEWART & OLSTEIN, 6 Becker Farm Road, Roseland, NJ, 07068, US

(21) Appl. No.: 10/177,629

(22) Filed: Jun. 19, 2002

Related U.S. Application Data
(60) Provisional application No. 60/299,345, filed on Jun. 19, 2001.

(51) Int. Cl. C12Q 1/68; C12P 19/34

(52) U.S. Cl. 435/6; 435/91.2

(57) ABSTRACT

Methods for the amplification of signals generated from target molecules using a plurality of bridging layers of detector molecules and rolling circle amplification of oligonucleotide sequences are disclosed, along with methods of using these together with solid supports, such as on a microarray.

This application claims the benefit of U.S. Provisional Application Serial No. 60/299,345, filed Jun. 19, 2001, the disclosure of which is hereby incorporated by reference in its entirety.

### FIELD OF THE INVENTION

The present invention relates to processes for enhanced signal amplification of molecular structures using rolling circle amplification.

### BACKGROUND OF THE INVENTION

A means of amplifying circular target DNA molecules is of value because such amplified DNA is frequently used in subsequent methods including DNA sequencing, cloning, mapping, genotyping, generation of probes, and diagnostic identification.

Heretofore, several useful methods were developed that permit amplification of nucleic acids. Most were designed around the amplification of selected DNA targets and/or probes, including the polymerase chain reaction (PCR), ligase chain reaction (LCR), self-sustained sequence replication (3SR), nucleic acid sequence based amplification (NASBA), strand displacement amplification (SDA), and amplification with Qβ replicase (Birkenmeyer and Mushahwar, J. Virological Methods, 35:117-126 (1991); Landegren, Trends Genetics, 9:199-202 (1993)).

In addition, several methods have been employed to amplify circular DNA molecules such as plasmids or DNA from bacteriophage such as M13. One has been propagation of these molecules in suitable host strains of E. coli, followed by isolation of the DNA by well-established protocols (Sambrook, J., Fritsch, E. F., and Maniatis, T. Molecular Cloning, A Laboratory Manual, 1989, Cold Spring Harbor Laboratory Press, Cold Spring Harbor, N.Y.). PCR has also been a frequently used method to amplify defined sequences in DNA targets such as plasmids and DNA from bacteriophage such as M13 (PCR Protocols, 1990, Ed. M. A. Innis, D. H. Gelfand, J. J. Sninsky, Academic Press, San Diego.)

As an improvement on these methods, linear rolling circle amplification (LRCA) uses a primer annealed to a circular target DNA molecule and DNA polymerase is added. The amplification target circle (ATC) forms a template on which new DNA is made, thereby extending the primer sequence as a continuous sequence of repeated sequences complementary to the circle but generating only about several thousand copies per hour. An improvement on LRCA is the use of exponential RCA (ERCA), with additional primers that anneal to the replicated complementary sequences to provide new centers of amplification, thereby providing exponential kinetics and increased amplification. Exponential rolling circle amplification (ERCA) employs a cascade of strand displacement reactions, also referred to as HRCA (Lizardi, P. M. et al. Nature Genetics, 19, 225-231 (1998)). However, all such methods are designed around amplifying a polynucleotide sequence as a means of detecting an oligonucleotide or polynucleotide target rather than use of the amplification procedure for amplifying a signal regardless of the molecular nature of the target.

### BRIEF SUMMARY OF THE INVENTION

In one aspect, the present invention relates to a process for amplifying a signal from a molecular target comprising:

(a) contacting a target molecule, having a target site (TS), with a target detector molecule having a detector site (DS) and a detector target site (DTS), wherein said contacting occurs under conditions promoting the binding of said target site to said detector site to form a target-detector complex (TDC);

(b) contacting the TDC of (a) with a plurality of primer detector molecules, each primer detector molecule having a target detector site (TDS) and a primer site, said primer site comprising an oligonucleotide primer (P) sequence, under conditions promoting binding of the TDC to said TDS to form a layered target-detector-primer (LTDP) complex comprising a plurality of primer detector molecules bound to each target-detector complex;

(c) contacting the layered target detector primer (LTDP) complex of (b) with an amplification target circle (ATC) comprising at least one primer complementary site (P′) having a nucleotide sequence complementary to the sequence of the primer site (P) of the primer detector molecule of (b) under conditions promoting hybridization of P′ and P to form a target-detector-primer (TDP) complex;

(d) contacting the TDP complex of (c) with an enzyme that promotes rolling circle amplification of said primer (P) in the presence of a plurality of deoxynucleoside triphosphates (dNTPs),

thereby generating a labeled tandem sequence polynucleotide (TS-DNA) as an extension product of said primer.

In preferred embodiments thereof, the detector site (DS) and said detector target site (DTS) are structurally similar, or the detector site (DS) and said detector target site (DTS) are structurally identical or the detector site (DS) and said detector target site (DTS) are structurally different. In another preferred embodiment, the target molecule comprises a detectable marker.

In an additional preferred embodiment, the target molecule comprises a member selected from the group consisting of an oligonucleotide, a protein, a carbohydrate, a lipid and a small organic molecule, most preferably an oligonucleotide, especially where the oligonucleotide is a biotinylated oligonucleotide.

Another specific embodiment is one where the target molecule comprises biotin. Also preferred is where the target molecule is attached to a solid support, preferably glass or plastic, especially where the solid support is part of a microarray.

Also preferred are embodiments wherein the target detector molecule comprises streptavidin and/or the primer detector molecule (PD) comprises biotin and/or where the primer detector molecule (PD) comprises an antibody, preferably wherein said antibody is a biotinylated antibody or and anti-avidin antibody.

In another preferred embodiment, step (a) is carried out more than once, preferably n times, prior to step (b) wherein n is at least 2 and wherein in the repeated steps the detectable target molecule is the target detector complex (TDC) formed from a step (a). In preferred embodiments thereof, n is 2, 3, or 4.

A preferred embodiment of the methods of the invention encompass cases where the target molecule comprises an oligonucleotide, a protein, a carbohydrate, a lipid or a small organic molecule, and/or where the target molecule comprises biotin. In a preferred embodiment, this target molecule is an oligonucleotide, especially a biotinylated oligonucleotide. The primer detector molecule may also comprise biotin. In other such embodiments, the primer detector molecule (PD) comprises an antibody, preferably a biotinylated antibody or an anti-avidin antibody. In a most preferred embodiment, where step (a) is repeated at least once, the target detector molecule is streptavidin for all odd numbered rounds of step (a). In one such preferred embodiment, the streptavidin comprises a label, most preferably a fluorescent label, especially one of the group Cy2, Cy3, Cy3.5, Cy5, Cy5.5, fluorescein, 5,6-carboxymethyl fluorescein, Texas red, nitrobenz-2-oxa-1,3-diazol-4-yl (NBD), coumarin, dansyl chloride, or rhodamine. In a highly preferred embodiment, the label is a radiolabel.

In an alternative preferred embodiment, where step(a) is repeated at least once, the target detector molecule comprises biotin for all even numbered rounds of step (a). the target detector molecule comprises an antibody for all even numbered rounds of step (a), preferably wherein said antibody is a biotinylated antibody or an anti-avidin antibody, most preferably a biotinylated-anti-avidin antibody.

In any of the methods of the invention, the enzyme of step (d) is selected from the group consisting of bacteriophage φ29 DNA polymerase, Tts DNA polymerase, phage M2 DNA polymerase, phage Φ-PRD1 DNA polymerase, VENT™ DNA polymerase, Klenow fragment of DNA polymerase I, T5 DNA polymerase, PRD1 DNA polymerase, T4 DNA polymerase holoenzyme, T7 native polymerase and Bst polymerase, preferably bacteriophage φ29 DNA polymerase, most preferably wherein said DNA polymerase does not exhibit 3′,5′-exonuclease activity.

In this most preferred embodiment, the DNA polymerase is selected from the group consisting of Taq, Tfl, and Tth DNA polymerase, Eukaryotic DNA polymerase alpha, and DNA polymerases that have been modified to eliminate a 3′-5′ exonuclease activity such as exo (−) versions of φ29 DNA polymerase, Klenow fragment, Vent and Pfu DNA polymerases.

In another preferred embodiment of the methods of the invention, the DNA polymerase is a reverse transcriptase.

In an additional preferred embodiment, the amplification target circle, or ATC, is RNA and the DNA polymerase is a reverse transcriptase. Alternatively, a linear DNA target is used instead of said ATC.

In the methods of the invention, the dNTPs are from the group consisting of dTTP, dCTP, dATP, dGTP, dUTP, a naturally occurring dNTP different from the foregoing, an analog of a dNTP, and a dNTP having a universal base, or any combinations of these. These may themselves may be linked to a label, such as a fluorescent or other detectable chemical label, or a radiolabel, where one or more atoms of the deoxynucleoside triphosphate is radioactive.

The present invention advantageously provides for enhanced signal detection using rolling circle amplification along with layered detection schemes, such as bridging layers prior to or following the rolling circle process as well as processes for signal amplification of any type of molecular target, such as a polynucleotide, a protein, a carbohydrate or a lipid or any other type of molecular structure that can be incorporated into a detectable target and linked to a molecule that can support rolling circle amplification.

In preferred embodiments, the present invention relates to the use of one, two, three or more bridging layers of detector molecules followed by rolling circle amplification wherein the target molecule to be detected is one that is optionally attached to a solid support, such as glass or plastic, and which support may be part of a microarray system, such as one containing a large number of molecular targets of varying molecular structure and identity.

The present invention accomplishes signal amplification by utilizing one or more rounds of rolling circle amplification followed by addition of one or more bridging layers containing detectable labels for amplified signal detection.

### BRIEF DESCRIPTION OF THE DRAWINGS

FIG. 1 shows a schematic diagram of a process of the invention utilizing a seeded-iRCAT (immuno-rolling circle amplification) procedure wherein seeding is carried out by one or more cycles of (Cy5-streptavidin, washing, biotin-antiavidin antibody, and washing) to form bridged layers for target detection. RCA is then conducted using an anti-biotin-primer conjugate wherein the primer can be extended on an amplification target circle (ATC) template to form a tandem sequence DNA (TS-DNA) product of repeated sequences present in the primer and complementary to the ATC. In this example, the target is a biotynylated oligonucleotide.

FIG. 2A shows the results of using various targets with the methods of the invention on a microarray. FIG. 2B shows fold amplification for each of the runs in FIG. 2A with the particular allele presented along the abscissa.

FIG. 3A shows the application of layered RCA of the invention to genomic DNA genotyping. FIG. 3B shows the location of markers and alleles for the run of FIG. 3A.

FIG. 4 shows an example of genotyping using the process of the invention on a microarray system.

### DETAILED DESCRIPTION OF THE INVENTION

In one general aspect, the present invention relates to a process for amplifying a signal from a molecular target comprising:

(a) contacting a target molecule, having a target site (TS), with a target detector molecule having a detector site (DS) and a detector target site (DTS), wherein said contacting occurs under conditions promoting the binding of said target site to said detector site to form a target-detector complex (TDC);

(b) contacting the TDC of (a) with a plurality of primer detector molecules, each primer detector molecule having a target detector site (TDS) and a primer site, said primer site comprising an oligonucleotide primer (P) sequence, under conditions promoting binding of the TDC to said TDS to form a layered target-detector-primer (LTDP) complex comprising a plurality of primer detector molecules bound to each target-detector complex;

(c) contacting the layered target detector primer (LTDP) complex of (b) with an amplification target circle (ATC) comprising at least one primer complementary site (P′) having a nucleotide sequence complementary to the sequence of the primer site (P) of the primer detector molecule of (b) under conditions promoting hybridization of P′ and P to form a target-detector-primer (TDP) complex;

(d) contacting the TDP complex of (c) with an enzyme that promotes rolling circle amplification of said primer (P) in the presence of a plurality of deoxynucleoside triphosphates (dNTPs),

thereby generating a labeled tandem sequence polynucleotide (TS-DNA) as an extension product of said primer.

In preferred embodiments, the detector site (DS) and the detector target site (DTS) are structurally similar, or possibly identical, or may be structurally different from each other.

Of course, such a method could be carried out using more than one round of the process, such as where steps (b) and (c) are repeated one or more times following step (a) and prior to effecting step (d). Alternatively, step (a) may be carried out more than once before step (b) is effected. In a preferred embodiment of such a method, step (a) is repeated once so that the method comprises step (a) being carried out twice.

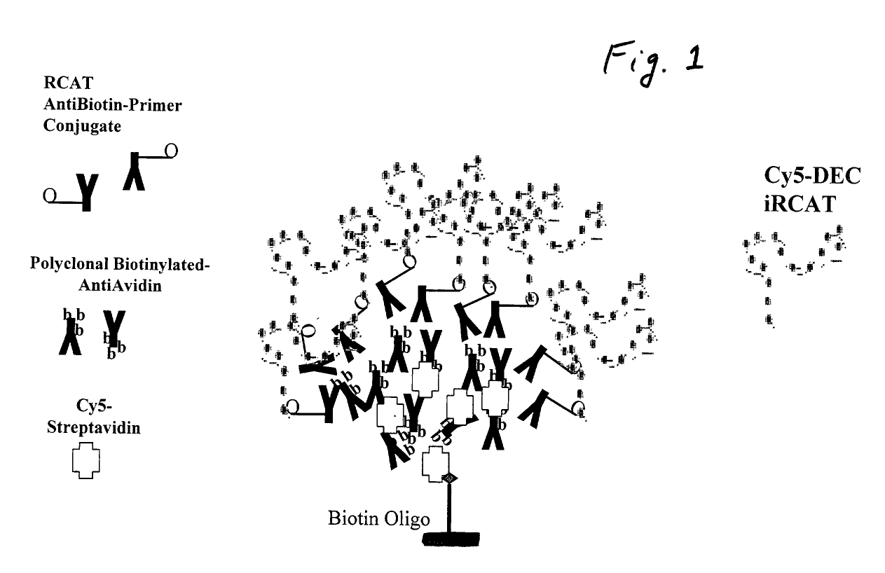

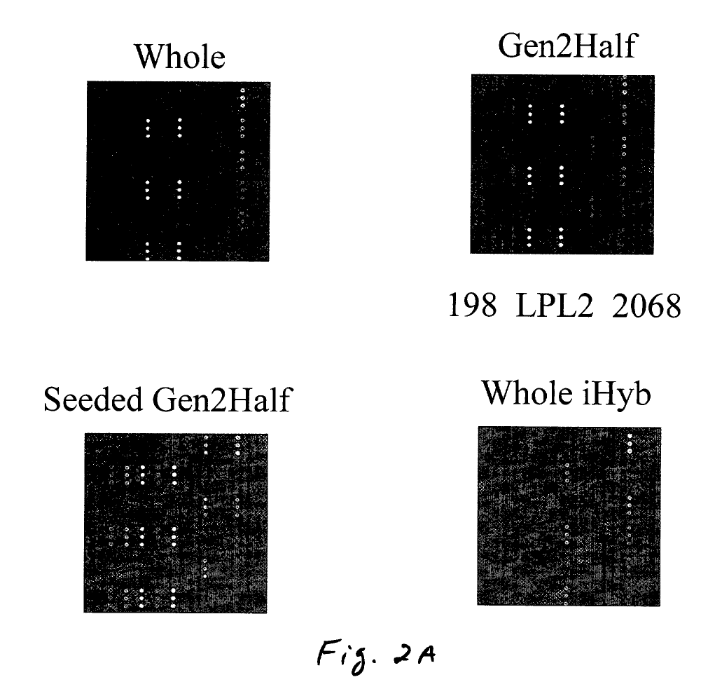

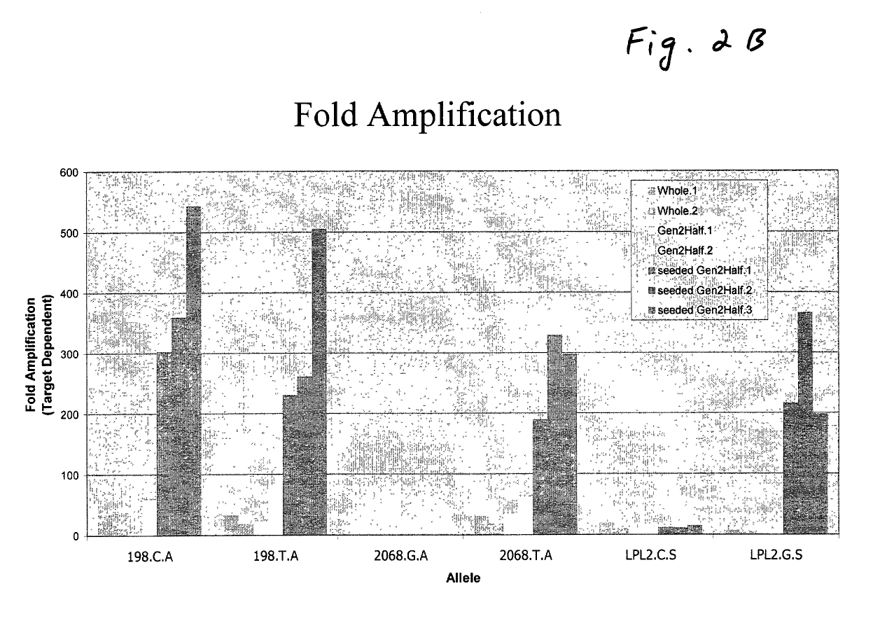

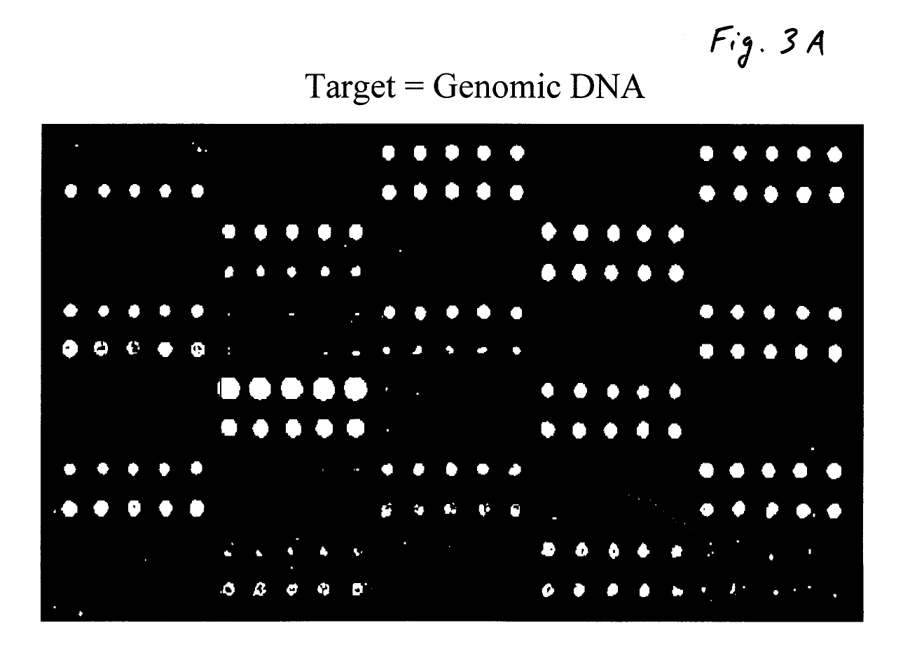

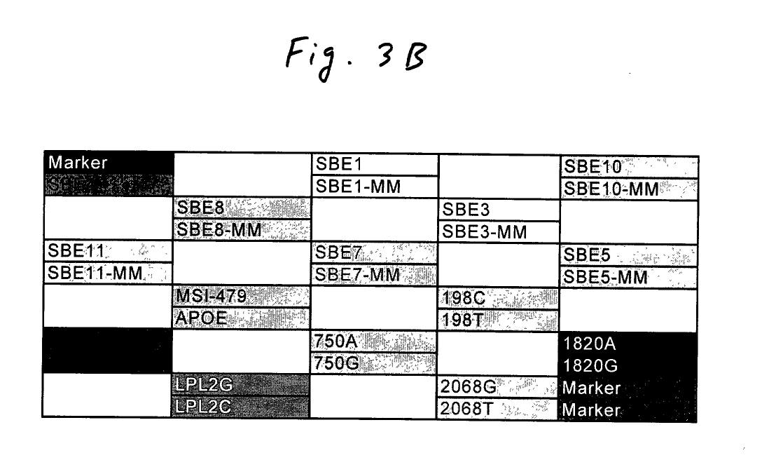

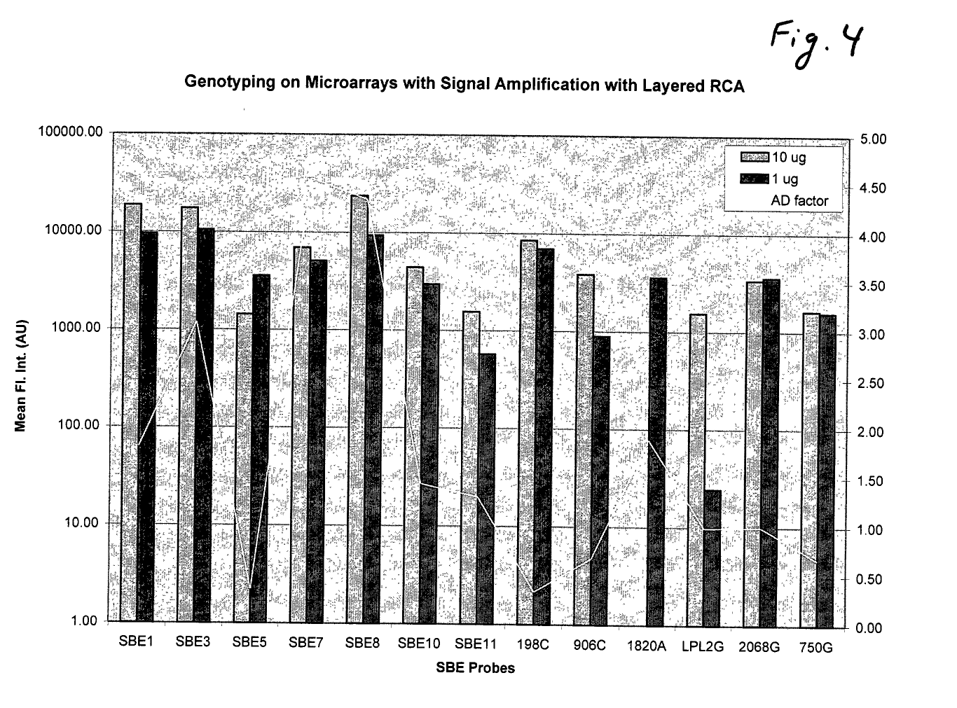

Of course, in applying the methods of the invention to target detection, the target is in no way limited to any particular kind of chemical structure but may include any type of molecule that will bind to a detectable marker, such as biotin. Thus, any molecule capable of being biotinylated can represent a detectable target whose signal can be readily amplified by the RCA-based processes disclosed herein. In specific embodiments, such target molecule can include an oligonucleotide, a protein, a carbohydrate or a lipid, such as the biotinylated oligonucleotide used in the procedure depicted in FIG. 1.

In one embodiment, this process is carried out in solution or suspension. In another embodiment, the target molecule is attached to a solid support, preferably one made of glass or plastic. Such support may be part of a microarray.

In one preferred embodiment, the target detector molecule comprises streptavidin. In another, the primer detector molecule (PD) comprises biotin or an antibody or both. Thus, said antibody may be a biotinylated antibody or an antiavidin antibody or a biotinylated-antiavidin antibody.

In any of the processes disclosed herein, where any reactant comprises streptavidin, the streptavidin may be labeled, such as by a fluorescent structure or radioactive atom.

Examples of suitable fluorescent labels include CyDyes such as Cy2, Cy3, Cy3.5, Cy5, And Cy5.5, available from Amersham Pharmacia Biotech (U.S. Pat. No. 5,268,486). Further examples of suitable fluorescent labels include fluorescein, 5,6-carboxymethyl fluorescein, Texas red, nitrobenz-2-oxa-1,3-diazol-4-yl (NBD), coumarin, dansyl chloride, and rhodamine. Preferred fluorescent labels are fluorescein (5-carboxyfluorescein-N-hydroxysuccinimide ester) and rhodamine (5,6-tetramethyl rhodamine). These can be obtained from a variety of commercial sources, including Molecular Probes, Eugene, Oreg. and Research Organics, Cleveland, Ohio.

In addition, the detector molecules, whether target detectors or primer detectors, may also comprise an antibody, which term is used in its most general sense. With the advent of methods of molecular biology and recombinant technology, it is now possible to produce antibody molecules by recombinant means and thereby generate gene sequences that code for specific amino acid sequences found in the polypeptide structure of the antibodies. Such antibodies can be produced by either cloning the gene sequences encoding the polypeptide chains of said antibodies or by direct synthesis of said polypeptide chains, with in vitro assembly of the synthesized chains to form active tetrameric (H₂L₂) structures with affinity for specific epitopes and antigenic determinants. This has permitted the ready production of antibodies having sequences characteristic of neutralizing antibodies from different species and sources.

Regardless of the source of the antibodies, or how they are recombinantly constructed, or how they are synthesized, in vitro or in vivo, using transgenic animals, such as cows, goats and sheep, using large cell cultures of laboratory or commercial size, in bioreactors or by direct chemical synthesis employing no living organisms at any stage of the process, all antibodies have a similar overall 3 dimensional structure. This structure is often given as H₂L₂ and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity.

The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure.

The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The accepted CDR regions have been described by Kabat et al, J. Biol. Chem. 252:6609-6616 (1977). The numbering scheme is shown in the figures, where the CDRs are underlined and the numbers follow the Kabat scheme.

In all mammalian species, antibody polypeptides contain constant (i.e., highly conserved) and variable regions, and, within the latter, there are the CDRs and the so-called “framework regions” made up of amino acid sequences within the variable region of the heavy or light chain but outside the CDRs.

The antibodies useful in practicing the processes of the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain. Such antibodies may also include fragments, such as Fab and F(ab₂)′ fragments, capable of reacting with and binding to any of the polypeptides disclosed herein as being receptors.

Depending upon the size of the amplification target circle (ATC) used in the RCA step (step (c) in the process described above), as well as the structure of the primers, and the DNA polymerase used, the process of the invention achieves an extremely high degree of signal amplification that can be further optimized at the level of primer extension by utilizing different DNA polymerases, dNTPs and Mg²⁺.

In accordance with the processes disclosed herein, the present invention relates to a process for signal amplification by amplifying nucleic acid sequences, comprising contacting a primer-bearing detector molecule (TDP), such as the antibiotin-primer conjugate shown in FIG. 1, with one or more amplification target circles (ATCs), a DNA polymerase and multiple deoxynucleoside triphosphates, under conditions wherein said ATC, bearing a primer complementary sequence (P′) with a nucleotide sequence complementary to said primer sequence of the TDP complex, binds to the TDP complex and wherein conditions promote replication of the amplification target circle by extension of the primers to form multiple tandem sequence DNA (TS-DNA) products, the latter being comprised of repeated sequences of polynucleotide complementary to the sequence of the ATC template.

In some circumstances it may be desirable to quantitatively determine the extent of amplification occurring and/or the amount of TS-DNA being formed or, in some circumstances, to be able to measure in a discriminating fashion the relative quantities of amplification target circles being formed where the ATCs of the starting mixture are not uniform in structure and/or size. In such instances, the present invention works well with any number of standard detection schemes, such as where special deoxynucleoside triphosphates (dNTPs) are utilized that make it easier to do quantitative measurements. The most common example is where such nucleotide substrates are radiolabeled or have attached thereto some other type of label, such as a fluorescent label or the like. Again, the methods that can be employed in such circumstances are many and the techniques involved are standard and well known to those skilled in the art. Thus, such detection labels include any molecule that can be associated with amplified nucleic acid, directly or indirectly, and which results in a measurable, detectable signal, either directly or indirectly. Many such labels for incorporation into nucleic acids or coupling to nucleic acid probes are known to those of skill in the art. General examples include radioactive isotopes, fluorescent molecules, phosphorescent molecules, enzymes, antibodies, and ligands. For example, any of the already mentioned fluorescent labels may be used.

Labeled nucleotides are a preferred form of detection label since they can be directly incorporated into the products of RCA during synthesis. Examples of detection labels that can be incorporated into amplified DNA include nucleotide analogs such as BrdUrd (Hoy and Schimke, Mutation Research, 290:217-230 (1993)), BrUTP (Wansick et al., J. Cell Biology, 122:283-293 (1993)) and nucleotides modified with biotin (Langer et al., Proc. Natl. Acad. Sci. USA, 78:6633 (1981)) or with suitable haptens such as digoxygenin (Kerkhof, Anal. Biochem., 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (BUDR triphosphate, Sigma), and a preferred nucleotide analog detection label is Biotin-16-uridine-5′-triphosphate (Biotin-16-dUTP, Boehringher Mannheim). Radiolabels are especially useful for the amplification methods disclosed herein. Thus, such dNTPs may incorporate a readily detectable moiety, such as a fluorescent label as described herein.

The present invention provides a means to achieve signal amplification in a variety of methods. The goal is to amplify a signal that allows detection or characterization of a target molecule and the present invention provides a way to amplify DNA product and thereby signal intensity.

DNA polymerases useful in the rolling circle replication step of the processes of the invention must perform rolling circle replication of primed single-stranded circles (or each strand of a duplex substrate). Such polymerases are referred to herein as rolling circle DNA polymerases. For rolling circle replication, it is preferred that a DNA polymerase be capable of displacing the strand complementary to the template strand, termed strand displacement, and lack a 5′ to 3′ exonuclease activity. Strand displacement is necessary to result in synthesis of multiple tandem copies of the ATC. A 5′ to 3′ exonuclease activity, if present, might result in the destruction of the synthesized strand. It is also preferred that DNA polymerases for use in the disclosed method are highly processive. The suitability of a DNA polymerase for use in the disclosed method can be readily determined by assessing its ability to carry out rolling circle replication. Preferred rolling circle DNA polymerases are bacteriophage φ-29 DNA polymerase (U.S. Pat. Nos. 5,198,543 and 5,001,050 to Blanco et al.), phage M2 DNA polymerase (Matsumoto et al., Gene 84:247 (1989)), phage PRD1 DNA polymerase (Jung et al., Proc. Natl. Acad. Sci. USA 84:8287 (1987), and Zhu and Ito, Biochim. Biophys. Acta. 1219:267-276 (1994)), VENT.RTM. DNA polymerase (Kong et al., J. Biol. Chem. 268:1965-1975 (1993)), Klenow fragment of DNA polymerase I (Jacobsen et al., Eur. J. Biochem. 45:623-627 (1974)), T5 DNA polymerase (Chatterjee et al., Gene 97:13-19 (1991)), and T4 DNA polymerase holoenzyme (Kaboord and Benkovic, Curr. Biol. 5:149-157 (1995)). φ-29 DNA polymerase is most preferred. Equally preferred polymerases include T7 native polymerase, Bacillus stearothermophilus (Bst) DNA polymerase, Thermoanaerobacter thermohydrosulfuricus (Tts) DNA polymerase (U.S. Pat. No. 5,744,312), and the DNA polymerases of Thermus aquaticus, Thermus flavus or Thermus thermophilus. Equally preferred are the φ29-type DNA polymerases, which are chosen from the DNA polymerases of phages: φ29, Cp-1, PRD1 , φ15, φ21, PZE, PZA, Nf, M2Y, B103, SF5, GA-1, Cp-5, Cp-7, PR4, PR5, PR722, and L17. In a specific embodiment, the DNA polymerase is bacteriophage Φ29 DNA polymerase wherein the multiple primers are resistant to exonuclease activity and the target DNA is high molecular weight linear DNA.

Strand displacement during RCA, especially where duplex ATCs are utilized as templates, can be facilitated through the use of a strand displacement factor, such as a helicase. In general, any DNA polymerase that can perform rolling circle replication in the presence of a strand displacement factor is suitable for use in the processes of the present invention, even if the DNA polymerase does not perform rolling circle replication in the absence of such a factor. Strand displacement factors useful in RCA include BMRF1 polymerase accessory subunit (Tsurumi et al., J. Virology 67(12):7648-7653 (1993)), adenovirus DNA-binding protein (Zijderveld and van derVliet, J. Virology 68(2):1158-1164 (1994)), herpes simplex viral protein ICP8 (Boehmer and Lehman, J. Virology 67(2):711-715 (1993); Skaliter and Lehman, Proc. Natl. Acad. Sci. USA 91(22):10665-10669 (1994)), single-stranded DNA binding proteins (SSB; Rigler and Romano, J. Biol. Chem. 270:8910-8919 (1995)), and calf thymus helicase (Siegel et al., J. Biol. Chem. 267:13629-13635 (1992)).

The ability of a polymerase to carry out rolling circle replication can be determined by using the polymerase in a rolling circle replication assay such as those described in Fire and Xu, Proc. Natl. Acad. Sci. USA 92:4641-4645 (1995) and in Lizardi (U.S. Pat. No. 5,854,033, e.g., Example 1 therein).

In practicing the processes of the present invention, any of the processes may be carried out in suspension or may be carried out with the target molecule attached to a solid support. Many such structures are known in the literature and for such uses the target molecule can, of course, be any type of molecule that can be attached to a solid support.

This includes materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin films or membranes, beads, bottles, dishes, fibers, woven fibers, shaped polymers, particles and microparticles. A preferred form for a solid-state substrate is a glass slide or a microtiter dish (for example, the standard 96-well dish). Preferred embodiments utilize glass or plastic as the support. For additional arrangements, see those described in U.S. Pat. No. 5,854,033.

Methods for immobilization of different kinds of target molecules are known in the literature and will not be described in detail herein. However, for by way of example, where the target molecule comprises an oligonucleotide, such as a biotinylated oligonucleotide (see, for example, FIG. 1) oligonucleotides can be attached to solid-state substrates using attachment methods as described by Pease et al., Proc. Natl. Acad. Sci. USA 91(11):5022-5026 (1994). A preferred method of attaching oligonucleotides to solid-state substrates is described by Guo et al., Nucleic Acids Res. 22:5456-5465 (1994). As a result, any molecule capable of being attached to an oligonucleotide can therefore be attached to a solid state substrate, such as a microarray, using these methods.

Oligonucleotides useful in forming the primers and amplification target circles of the present invention, such as in the formation of microarrays for use with the present invention, can be synthesized using established oligonucleotide synthesis methods to afford any desired sequence of nucleotides. Methods of synthesizing oligonucleotides are well known in the art. Such methods can range from standard enzymatic digestion followed by nucleotide fragment isolation (see for example, Sambrook, et al., Molecular Cloning: A Laboratory Manual, Third Edition, Cold Spring Harbor, N.Y., (2000), Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

In addition, procedures for the synthesis of oligonucleotides of desired sequence and containing phosphorothioate diesters by chemical sulfurization are well-established. The solid phase synthesis of random primers employs one or several specifically placed internucleotide phosphorothioate diesters at the 3′-end. Phosphorothioate triesters can be introduced by oxidizing the intermediate phosphite triester obtained during phosphoramidite chemistry with 3H-1, 2-benzodithiol-3-one 1,1 dioxide or Beaucage reagent to generate pentavalent phosphorous in which the phosphorothioate triester exists as a thione. The thione formed in this manner is stable to the subsequent oxidation steps necessary to generate internucleotidic phosphodiesters. (lyer, R. P., Egan, W., Regan, J. B., and Beaucage, S. L. J. Am. Chem. Soc., 112: 1253 (1990), and lyer, R. P., Philips, L. R., Egan, W., Regan, J. B., and Beaucage, S. L. J. Org. Chem., 55:4693 (1990))

In practicing the processes of the present invention, any number of bridging layers may be utilized before or after the rolling circle step. Thus, one, two, three or more layers may be added to amplify signal generation prior to rolling circle amplification using the target-detector-primer (TDP) complex and complementary amplification target circle.

For example, the process may be carried out by utilizing multiple rounds of step (a) wherein the target molecule of each succeeding round of step (a) is the target detector complex of the previous round of step (a).

Thus, the present invention relates to a process as disclosed hereinabove wherein step (a) is carried out n times prior to step (b) wherein n is at least 2 and wherein in the repeated steps the detectable target molecule is the target detector complex (TDC) formed from a step (a). In preferred embodiment, n is equal to 2, 3, 4 or more.

In preferred embodiments of such a process, the target detector molecule is streptavidin for all odd numbered rounds of step (a). In other preferred embodiments, the target detector molecule comprises biotin for all even numbered rounds of step (a). In yet other preferred embodiments, the target detector molecule comprises an antibody for all even numbered rounds of step (a), preferably where said antibody is a biotinylated antibody or is an anti-avidin antibody, most preferably a biotinylated-anti-avidin antibody.

Thus, the present invention also contemplates the use of additional layers prior to rolling circle amplification, as would be formed by the additional rounds of step (a) and which are depicted by way of a limited example in FIG. 1.

One such embodiment of a process employing multiple rounds of step (a) is a process for amplifying a signal from a molecular target comprising:

(a) contacting a detectable target molecule, having a first target site (TS-1), with a plurality of first detector molecules, each having a first detector site (DS-1) and a second target site (TS-2), under conditions promoting the binding of said TS-1 to at least one DS-1 to form a first target-detector (TD-1) complex;

(b) contacting the TD-1 of (a) with a plurality of second detector molecules, each having a second detector site (DS-2) and a third target site (TS-3), under conditions promoting the binding of the TS-2 of the first detector complex to said DS-2 to form a second target-detector (TD-2) complex;

(c) contacting the TD-2 of (b) with a plurality of third detector molecules, each having a third detector site (DS-3) and a fourth target site (TS-4), under conditions promoting the binding of the TS-3 of the second detector complex to said DS-3 to form a third target-detector (TD-3) complex;

(d) contacting the TD-3 of (c) with a plurality of fourth detector molecules, each having a fourth detector site (DS-4) and a primer site, said primer site comprising an oligonucleotide primer (P) sequence suitable for rolling circle amplification, under conditions promoting the binding of the TS-4 of the third detector complex (TD-3 of (c) to said DS-4 to form a target-detector-primer (TDP) complex;

(e) contacting the target detector primer (TDP) complex of (d) with an amplification target circle (ATC) comprises at least one primer complementary sequence (P′) which is complementary to the oligonucleotide primer (P) of the fourth detector molecule of (d) under conditions promoting the hybridization of said complementary primer sequences to said oligonucleotide primers forming a P-P′ hybridized complex;

(f) contacting the complex of (e) with an enzyme that promotes rolling circle amplification of said primer (P) in the presence of a plurality of labeled dNTPs,

thereby forming a labeled tandem sequence polynucleotide (TS-DNA) as an extension product of said primer.

This process thus employs three rounds of step (a). Similar methodologies and reactants as are described hereinabove may be used with this embodiment as well.

Additional layers may further be utilized by adding a fourth round of step (a). In accordance therewith, the present invention also relates to a process for amplifying a signal from a molecular target comprising:

(a) contacting a target molecule, such as a detectable target molecule, having a first target site (TS-1), with a plurality of first detector molecules, each having a first detector site (DS-1) and a second target site (TS-2), under conditions promoting the binding of said TS-1 to at least one DS-1 to form a first target-detector (TD-1) complex;

(b) contacting the TD-1 of (a) with a plurality of second detector molecules, each having a second detector site (DS-2) and a third target site (TS-3), under conditions promoting the binding of the TS-2 of the first detector complex to said DS-2 to form a second target-detector (TD-2) complex;

(c) contacting the TD-2 of (b) with a plurality of third detector molecules, each having a third detector site (DS-3) and a fourth target site (TS-4), under conditions promoting the binding of the TS-3 of the second detector complex to said DS-3 to form a third target-detector (TD-3) complex;

(d) contacting the TD-3 of (c) with a plurality of fourth detector molecules, each having a fourth detector site (DS-4) and a fifth target site (TS-5), under conditions promoting the binding of the TS-4 of the third detector complex to said DS-4 to form a fourth target-detector (TD-4) complex,

(e) contacting the TD-4 of (d) with a plurality of fifth detector molecules, each having a fifth detector site (DS-5) and a primer site, said primer site comprising an oligonucleotide primer (P) sequence suitable for rolling circle amplification, under conditions promoting the binding of the TS-5 of the fourth detector complex (TD-4 of d) to said DS-5 to form a target-detector-primer (TDP) complex;

(f) contacting the target detector primer (TDP) complex of (e) with an amplification target circle (ATC) comprises at least one primer complementary sequence (P′) which is complementary to the oligonucleotide primer (P) of the fifth detector molecule of (c) under conditions promoting the hybridization of said complementary primer sequences to said oligonucleotide primers forming a P-P′ hybridized complex;

(g) contacting the complex of (e) with an enzyme that promotes rolling circle amplification of said primer (P) in the presence of a plurality of labeled dNTPs,

thereby forming a labeled tandem sequence polynucleotide (TS-DNA) as an extension product of said primer.

In preferred embodiments of this process, the third detector site (DS-3) and the fourth target site (TS-4) are structurally different, or structurally similar if not the same. In preferred embodiments, the detector molecule comprises streptavidin, and/or the detector molecule is an antibody, most preferably an antistreptavidin antibody (such as that depicted in FIG. 1). In another preferred embodiment, the fourth detector molecule comprises biotin.

What is claimed is:

1. A method for amplifying a signal from a molecular target comprising:
(a) contacting a target molecule, having a target site (TS), with a target detector molecule having a detector site (DS) and a detector target site (DTS), wherein said contacting occurs under conditions promoting the binding of said target site to said detector site to form a target-detector complex (TDC);
(b) contacting the TDC of (a) with a plurality of primer detector molecules, each primer detector molecule having a target detector site (TDS) and a primer site, said primer site comprising an oligonucleotide primer (P) sequence, under conditions promoting binding of the TDC to said TDS to form a layered target-detector-primer (LTDP) complex comprising a plurality of primer detector molecules bound to each target-detector complex;
(c) contacting the layered target detector primer (LTDP) complex of (b) with an amplification target circle (ATC) comprising at least one primer complementary site (P′) having a nucleotide sequence complementary to the sequence of the primer site (P) of the primer detector molecule of (b) under conditions promoting hybridization of P′ and P to form a target-detector-primer (TDP) complex;
(d) contacting the TDP complex of (c) with an enzyme that promotes rolling circle amplification of said primer (P) in the presence of a plurality of deoxynucleoside triphosphates (dNTPs),
thereby generating a labeled tandem sequence polynucleotide (TS-DNA) as an extension product of said primer.

2. The method of claim 1 wherein said detector site (DS) and said detector target site (DTS) are structurally similar.

3. The method of claim 1 wherein said detector site (DS) and said detector target site (DTS) are structurally identical.

4. The method of claim 1 wherein said detector site (DS) and said detector target site (DTS) are structurally different.

5. The method of claim 1 wherein said target molecule comprises a detectable marker.

6. The method of claim 1 wherein said target molecule comprises a member selected from the group consisting of an oligonucleotide, a protein, a carbohydrate, a lipid and a small organic molecule.

7. The method of claim 6 wherein said member is an oligonucleotide.

8. The method of claim 7 wherein said oligonucleotide is a biotinylated oligonucleotide.

9. The method of claim 1 wherein said target molecule comprises biotin.

10. The method of claim 1 wherein said target molecule is attached to a solid support.

11. The method of claim 10 wherein said solid support is selected from the group consisting of glass and plastic.

12. The method of claim 10 wherein said solid support is part of a microarray.

13. The method of claim 1 wherein said target detector molecule comprises streptavidin.

14. The method of claim 1 wherein said primer detector molecule (PD) comprises biotin.

15. The method of claim 1 wherein said primer detector molecule (PD) comprises an antibody.

16. The method of claim 15 wherein said antibody is a biotinylated antibody.

17. The method of claim 15 wherein said antibody is an anti-avidin antibody.

18. The method of claim 1 wherein step (a) is carried out n times prior to step (b) wherein n is at least 2 and wherein in the repeated steps the detectable target molecule is the target detector complex (TDC) formed from a step (a).

19. The method of claim 18 wherein n is 2.

20. The method of claim 18 wherein n is 3.

21. The method of claim 18 wherein n is 4.

22. The method of claim 18 wherein n is more than 4.

24. The method of claim 18 wherein said target molecule comprises a member selected from the group consisting of an oligonucleotide, a protein, a carbohydrate, a lipid and a small organic molecule.

25. The method of claim 18 wherein the target molecule comprises biotin.

26. The method of claim 24 wherein said member is an oligonucleotide.

27. The method of claim 26 wherein said oligonucleotide is a biotinylated oligonucleotide.

28. The method of claim 18 wherein said target molecule is attached to a solid support.

29. The method of claim 28 wherein said solid support is selected from the group consisting of glass and plastic.

30. The method of claim 28 wherein said solid support is part of a microarray.

31. The method of claim 18 wherein said primer detector molecule comprises biotin.

32. The method of claim 18 wherein said primer detector molecule (PD) comprises an antibody.

33. The method of claim 32 wherein the antibody is a biotinylated antibody.

34. The method of claim 32 wherein the antibody is an anti-avidin antibody.

35. The method of claim 18 wherein the target detector molecule is streptavidin for all odd numbered rounds of step (a).

36. The method of claims 13 or 35 wherein the streptavidin comprises a label.

37. The method of claim 36 wherein said label is a fluorescent label.

38. The method of claim 37 wherein said fluorescent label is selected from the group consisting of Cy2, Cy3, Cy3.5, Cy5, Cy5.5, fluorescein, 5,6-carboxymethyl fluorescein, Texas red, nitrobenz-2-oxa-1,3-diazol-4-yl (NBD), coumarin, dansyl chloride, and rhodamine.

39. The method of claim 37 wherein said label is a radiolabel.

40. The method of claim 18 wherein the target detector molecule comprises biotin for all even numbered rounds of step (a).

41. The method of claim 18 wherein the target detector molecule comprises an antibody for all even numbered rounds of step (a).

42. The method of claim 41 wherein said antibody is a biotinylated antibody.

43. The method of claim 41 wherein said antibody is an anti-avidin antibody.

44. The method of claim 41 wherein said antibody is a biotinylated-antiavidin antibody.

45. The method of claims 1 or 18 wherein said enzyme is selected from the group consisting of bacteriophage φ29 DNA polymerase, Tts DNA polymerase, phage M2 DNA polymerase, phage Φ-PRD1 DNA polymerase, VENT™ DNA polymerase, Klenow fragment of DNA polymerase 1, T5 DNA polymerase, PRD1 DNA polymerase, T4 DNA polymerase holoenzyme, T7 native polymerase and Bst polymerase.

46. The method of claim 45 wherein said DNA polymerase is bacteriophage φ29 DNA polymerase.

47. The method of claim 45 wherein said DNA polymerase does not exhibit 3′,5′-exonuclease activity.

48. The method of claim 47 wherein said DNA polymerase is selected from the group consisting of Taq polymerase, Tfl DNA polymerase, Tth DNA polymerase and Eukaryotic DNA polymerase alpha.

49. The method of claim 1 or 18 wherein said DNA polymerase is a reverse transcriptase.

50. The method of claim 1 or 18 wherein said ATC is RNA and said DNA polymerase is a reverse transcriptase.

51. The method of claim 1 or 18 wherein a linear DNA target is used instead of said ATC.

52. The method of claim 1 or 18 wherein said dNTP is a member selected from the group consisting of dTTP, dCTP, dATP, dGTP, dUTP, a naturally occurring dNTP different from the foregoing, an analog of a dNTP, and a dNTP having a universal base.

53. The method of claim 52 wherein at least one said dNTP is radiolabeled.

54. A method for amplifying a signal from a molecular target comprising:
(a) contacting a target molecule, such as a detectable target molecule, having a first target site (TS-1), with a plurality of first detector molecules, each having a first detector site (DS-1) and a second target site (TS-2), under conditions promoting the binding of said TS-1 to at least one DS-1 to form a first target-detector (TD-1) complex;
(b) contacting the TD-1 of (a) with a plurality of second detector molecules, each having a second detector site (DS-2) and a third target site (TS-3), under conditions promoting the binding of the TS-2 of the first detector complex to said DS-2 to form a second target-detector (TD-2) complex;
(c) contacting the TD-2 of (b) with a plurality of third detector molecules, each having a third detector site (DS-3) and a fourth target site (TS-4), under conditions promoting the binding of the TS-3 of the second detector complex to said DS-3 to form a third target-detector (TD-3) complex;
(d) contacting the TD-3 of (c) with a plurality of fourth detector molecules, each having a fourth detector site (DS-4) and a fifth target site (TS-5), under conditions promoting the binding of the TS-4 of the third detector complex to said DS-4 to form a fourth target-detector (TD-4) complex;
(e) contacting the TD-4 of (d) with a plurality of fifth detector molecules, each having a fifth detector site (DS-5) and a primer site, said primer site comprising an oligonucleotide primer (P) sequence suitable for rolling circle amplification, under conditions promoting the binding of the TS-5 of the fourth detector complex (TD-4 of d) to said DS-5 to form a target-detector-primer (TDP) complex;
(f) contacting the target detector primer (TDP) complex of (e) with an amplification target circle (ATC) comprises at least one primer complementary sequence (P′) which is complementary to the oligonucleotide primer (P) of the fifth detector molecule of (c) under conditions promoting the hybridization of said complementary primer sequences to said oligonucleotide primers forming a P-P′ hybridized complex;
(g) contacting the complex of (e) with an enzyme that promotes rolling circle amplification of said primer (P) in the presence of a plurality of labeled dNTPs,
thereby forming a labeled tandem sequence polynucleotide (TS-DNA) as an extension product of said primer.

55. The method of claim 54 wherein the third detector site (DS-3) and the fourth target site (TS-4) are structurally different.

56. The method of claim 54 wherein the third detector site (DS-3) and the fourth target site (TS-4) are structurally similar.

57. The method of claim 54 wherein the detector molecule comprises streptavidin.

58. The method of claim 54 wherein the detector molecule is an antibody.

59. The method of claim 58 wherein the antibody is an anti-streptavidin antibody.

60. The method of claim 54 wherein the detector molecule comprises biotin.
